(19) United States
(12) Patent Application Publication
(10) Pub. No.: US 2010/0206728 A1
(43) Pub. Date: Aug. 19, 2010

(54) BIOCHEMICAL TEST SYSTEM, MEASUREMENT DEVICE, AND BIOCHEMICAL TEST STRIP

(75) Inventors: Ying-Che Huang, Jhonghe City (TW); Mon Wen Yang, Hsinchu City (TW); Ming-Chang Hsu, Hsinchu City (TW); Thomas Y.S. Shen, Hsinchu City (TW)

Correspondence Address: SNELL & WILMER L.L.P. (Main), 400 EAST VAN BUREN, ONE ARIZONA CENTER, PHOENIX, AZ, 85004-2202, US

(73) Assignee: APEX BIOTECHNOLOGY CORP., Hsinchu City (TW)

(21) Appl. No.: 12/581,270

(22) Filed: Oct. 19, 2009

(30) Foreign Application Priority Data
Feb. 13, 2009 (TW) 098202095

(51) Int. Cl. G01N 33/50

(52) U.S. Cl. 204/403.02

(57) ABSTRACT

A biochemical test system, a measurement device, and a biochemical test strip are provided. The biochemical test strip includes an insulating substrate, a conductive layer, and at least one open-circuit part. The conductive layer is disposed on the insulating substrate and includes a plurality of electronic elements, wherein one end of the conductive layer is formed as a connection region. The at least one open-circuit part is disposed on at least one of the plurality of the electronic elements within the connection region. The type of the biochemical test strip is determined by the number and location of the at least one open-circuit part(s).

### CROSS REFERENCE TO RELATED APPLICATIONS

This application claims priority to Taiwan Patent Application No. 98202095 entitled “BIOCHEMICAL TEST SYSTEM, MEASUREMENT DEVICE, AND BIOCHEMICAL TEST STRIP,” filed on Feb. 13, 2009, which is incorporated herein by reference and assigned to the assignee herein.

### FIELD OF INVENTION

The present invention relates to a biochemical test system, a measurement device, and a biochemical test strip, and more particularly, to a biochemical test system, a measurement device, and a biochemical test strip having a self-identification function.

### BACKGROUND OF THE INVENTION

With the popularization of the self-testing products, the accuracy control of the biochemical index becomes more and more important for curing and preventing diabetes or other diseases. Although the conventional biochemical test strip and the system thereof are convenient to acquire the biochemical index, the accuracy may be unreliable due to the variations in biochemical test strip from batch to batch. Therefore, a code card is needed to calibrate the measurement device for most systems, as disclosed in U.S. Pat. No. 5,366,609. However, this method is very inconvenient for the user, and besides, the correction errors and the data measurement errors occur frequently because users may forget to insert the code card, use a wrong code card, or lose the code card.

To solve the inconvenience of using the code card, U.S. Pat. No. 6,814,844 disclosed a test strip with a bar code pattern formed on the substrate by laser ablation method, and WO 02/088739A1 disclosed a test strip with a bar code formed of invisible ink (such as ultraviolet ink or infrared ink). However, the systems using the bar code pattern for identification requires additional optical detectors for detection. Moreover, the reproduction and the accuracy highly depend on the surface condition of the target material, manufacture process, and the material of ink, which not only limits manners of fabrication, but also increases in the production cost.

In addition, Taiwan utility model patent No. M304662 disclosed a biochemical test system capable of being exempted from using a code card. The measurement device is equipped with several buttons which allows a user to enter specific English characters or numbers. These characters or numbers may be printed on the exterior package of the test strip (packing case, plastic box, manual, etc.) and correspond to a set of parameters stored in a correction unit of the measurement device. After entering the specific English characters or numbers, a microprocessor of the measurement device can select corresponding correction parameters to calibrate the measurement device.

Further, Taiwan patent application No. 97208206 disclosed a test strip capable of avoiding the need of the code card. A plurality of identifying elements are formed on one end of the test strip, and each identifying element can be punched selectively to construct various code patterns. However, there are a lot of limitations in this test strip, such as high precision requirement of punching process, high accuracy of alignment between the sensing terminals of a measurement device and the identifying elements of the test strip, and risk of breaking the test strip due to its tooth-like shape.

Accordingly, it is advantageous to have a biochemical test system capable of self-calibration, avoiding use of a code card correction, and keeping the production yield and the test accuracy.

### SUMMARY OF THE INVENTION

In view of the problems existing in the prior art, the present invention provides a biochemical test system, measurement device, and biochemical test strip capable of providing self-identification function, eliminating the use of a discrete code card, and reducing the production failure rate.

According to an aspect of the present invention, a biochemical test strip including an insulating substrate, a conductive layer, and at least one open-circuit part is provided. The conductive layer is disposed on the insulating substrate and includes a plurality of electronic elements, wherein one end of the conductive layer is formed as a connection region. The at least one open-circuit part is disposed on at least one of the plurality of the electronic elements within the connection region. The type of the biochemical test strip is determined by the number and location of the at least one open-circuit part.

According to another aspect of the present invention, a biochemical test system including a biochemical test strip and a measurement device is provided. The biochemical test strip includes an insulating substrate, a conductive layer disposed on the insulating substrate, and at least one open-circuit part, wherein the conductive layer has a plurality of electronic elements, and one end of the conductive layer is formed as a connection region. The at least one open-circuit part is disposed on at least one of the plurality of the electronic elements within the connection region. The type of the biochemical test strip is determined by number and location of the at least one open-circuit part. The measurement device includes a microprocessor and a connector, wherein the connector includes a plurality of connecting terminals corresponding to the plurality of electronic elements and the at least one open-circuit part respectively. The plurality of connecting terminals are configured to be coupled to the connection region for receiving a signal corresponding to the type of the biochemical test strip. The microprocessor is coupled to the connector for receiving the signal from the connector.

According to another aspect of the present invention, a measurement device is provided. The measurement device is used with a biochemical test strip, wherein the biochemical test strip includes an insulating substrate, a conductive layer disposed on the insulating substrate, and at least one open-circuit part. The conductive layer has a plurality of electronic elements, and one end of the conductive layer is formed as a connection region. The at least one open-circuit part is disposed on at least one of the plurality of the electronic elements within the connection region. The type of the biochemical test strip is determined by the number and location of the at least one open-circuit part. The measurement device includes a connector and a microprocessor. The connector includes a plurality of connecting terminals corresponding to the plurality of electronic elements and the at least one open-circuit part respectively, wherein the plurality of connecting terminals are configured to be coupled to the connection region for receiving a signal corresponding to the type of the biochemical test strip. The microprocessor is coupled to the connector for receiving the signal from the connector.

The other aspects of the present invention, part of which will be described in the following description, part of which will be apparent from description, can be known from the execution of the present invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE PICTURES

The foregoing aspects and many of the attendant advantages of this invention will become more readily appreciated as the same becomes better understood by reference to the following detailed description, when taken in conjunction with the accompanying pictures, wherein:

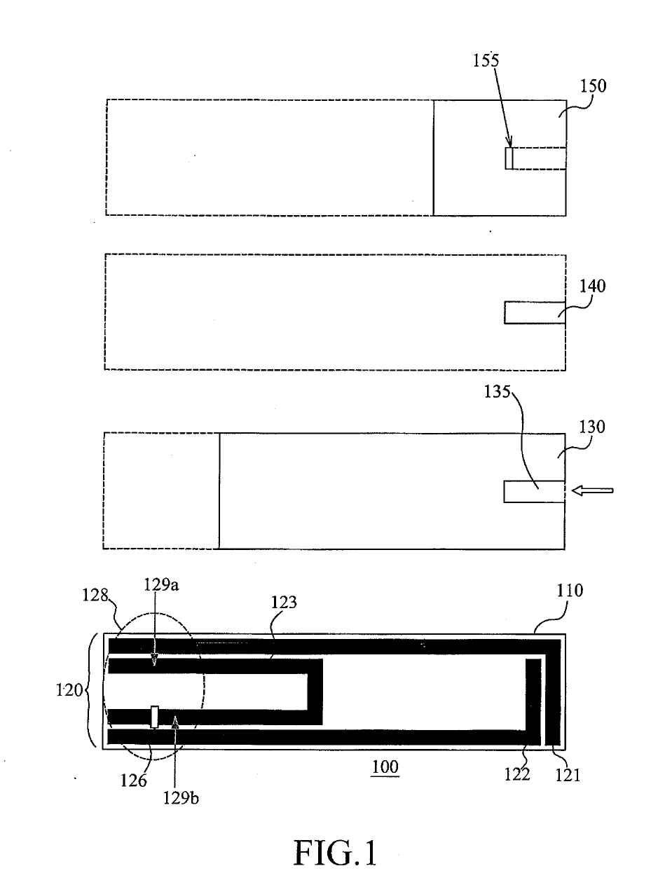

FIG. 1 illustrates an explosive view of a biochemical test strip according to an embodiment;

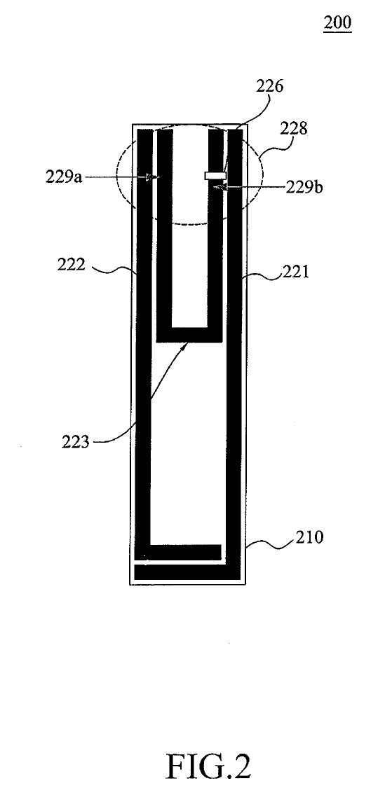

FIGS. 2-4 are the biochemical test strips according to different embodiments of the present invention;

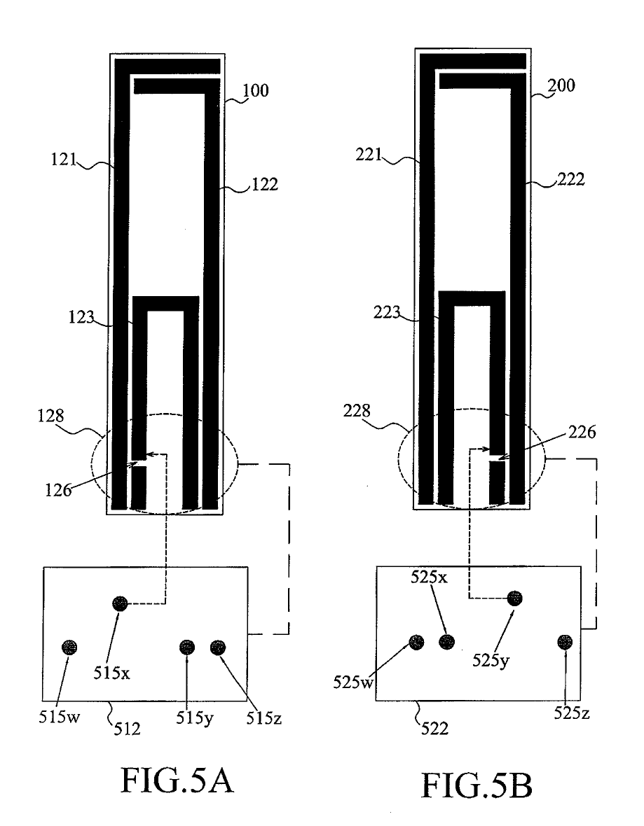

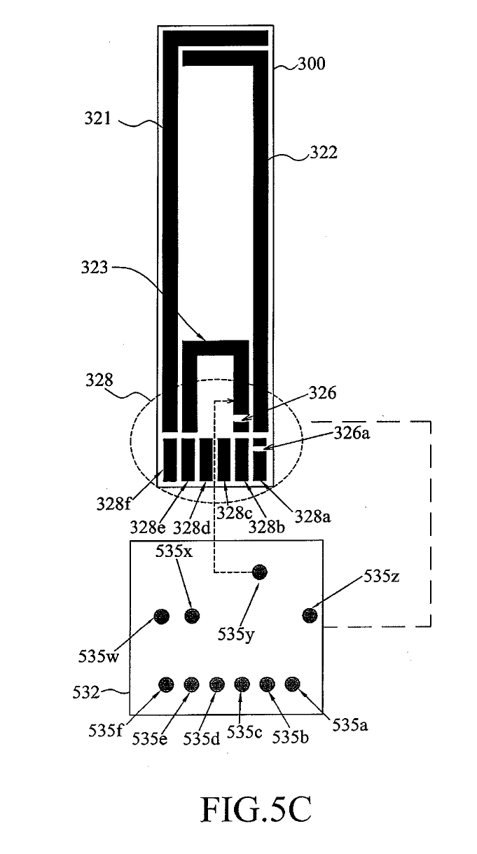

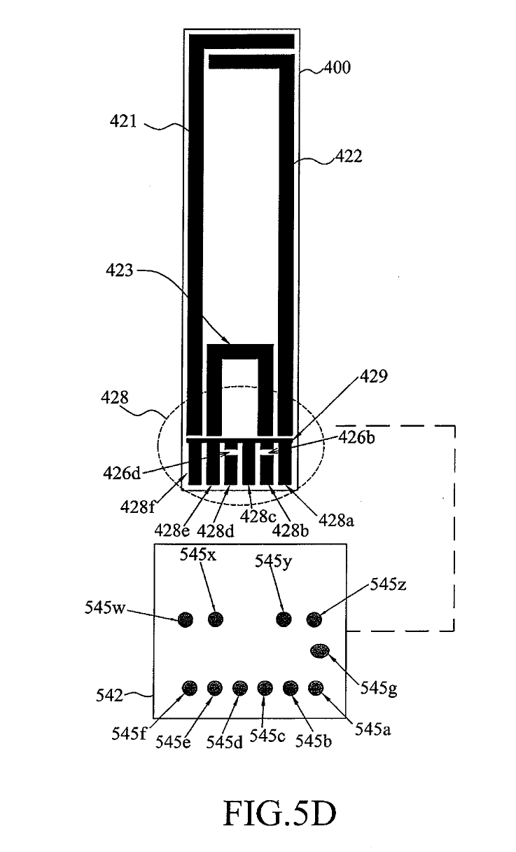

FIGS. 5A, 5B, 5C and 5D are illustrative diagrams showing the connecting terminals of the connectors according to different embodiments of the present invention respectively;

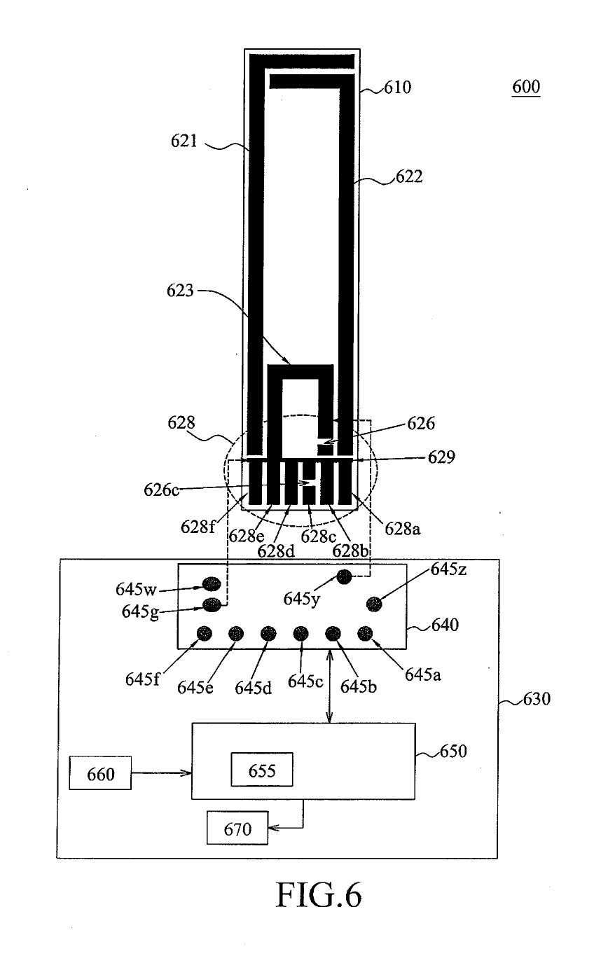

FIG. 6 is a block diagram of a biochemical test system according to an embodiment of the present invention; and

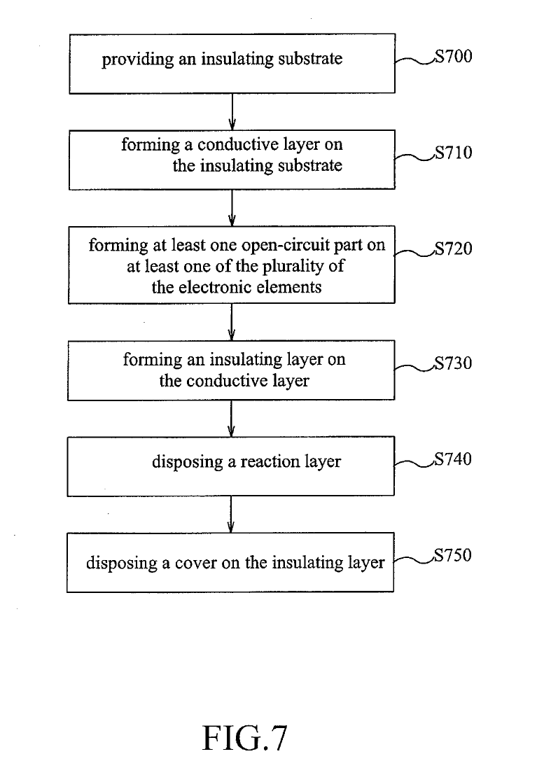

FIG. 7 is a flow chart of producing a biochemical test strip according to an embodiment of the present invention.

### DETAILED DESCRIPTION OF THE INVENTION

The present invention discloses a biochemical test system, a measurement device, and a biochemical test strip, which can eliminate the need of a discrete code card, provide easy operation for the user, prevent a user from forgetting to insert the code card or using a wrong code card, and reduce the possibility of errors during the production process. The present invention will be described more fully hereinafter with reference to the FIGS. 1-6. However, it should be noted that the features illustrated in the drawings are not necessarily drawn to scale, and like reference numerals represent the same or similar elements. The devices, elements, and methods in the following description are configured to illustrate the present invention, and should not be construed in a limiting sense.

FIG. 1 illustrates an explosive view of a biochemical test strip 100 according to an embodiment of the present invention. The biochemical test strip 100 of the present invention includes an insulating substrate 110, a conductive layer 120, an insulating layer 130, and a cover 150. The conductive layer 120 includes a plurality of electronic elements insulated from each other. In this embodiment, the conductive layer 120 includes a working electrode 121, a reference electrode 122, and a sensing electrode 123. One end of the conductive layer 120 can be defined as a connection region 128 which is configured to electrically connect to a measurement device (as 630 shown in FIG. 6). An open-circuit part 126 can be formed by applying an electrical damage on a part of one or more electronic elements within the connection region 128. In this embodiment, in response to the location of the open-circuit part 126, the sensing electrode 123 will be electrically connected to the measurement device at locations 129a and 129b.

The method of forming the open-circuit part 126 (i.e. the method of applying an electrical damage on the electronic elements) can be, for example, a laser etching method, a mechanical abrasion method, or a chemical etching method. For example, the open-circuit part 126 can be formed by electrolysis, exposure and development, planer process, drilling process, or other similar method. The laser etching process is a preferable method to precisely and finely make an electrical damage on the electronic element. The open-circuit part 126 can either penetrate or not penetrate the insulating substrate 110, and the number and the shape thereof need not be limited by the present invention. Simultaneously, the number and the location of the open-circuit part 126 can determine whether each of the electronic elements on the biochemical test strip 100 is capable of electrically connecting with a measurement device. Therefore, different types of the biochemical test strips have different types of electrical damage, whereby the measurement device can recognize the type of the biochemical test strip.

The insulating substrate 110 is electrically insulating and can be made of materials including, but not limited to, polyvinylchloride (PVC), glass fiber (FR-4), polyester, bakelite, polyethylene terephthalate (PET), Polycarbonate (PC), polypropylene (PP), polyethylene (PE), polystyrene (PS), or ceramic material.

The conductive layer 120 can be any known conductive material, such as carbon paste, gold-silver paste, copper paste, carbon/silver paste, other similar material, or the combination thereof. In an embodiment, the conductive layer 120 includes a conductive silver paste layer and a conductive carbon paste layer disposed on the conductive silver paste layer. In the embodiment shown in FIG. 1, the sensing electrode 123 is a [symbol] -shaped electrode and electrically insulated from the working electrode 121 and the reference electrode 122. In another embodiment, the sensing electrode 123 can be replaced by a set of sensing electrodes connecting by a resistor, as disclosed in Taiwan patent application number 94146334, which is incorporated herein by reference. In this embodiment, the sensing electrode 123 is disposed between the working electrode 121 and the reference electrode 122 and configured to detect an electrical connection between the biochemical test strip 100 and a measurement device (as 630 shown in FIG. 6). When the biochemical test strip 100 is inserted into the measurement device, a loop is formed between the sensing electrode 123 and the measurement device to initiate the measurement device. In the embodiment shown in FIG. 1, if the biochemical test strip 100 is not inserted into its corresponding measurement device correctly, the sensing electrode 123 may be unable to form a loop due to the presence of the open-circuit part 126, such that the measurement device may be unable to be initiated. It should be noted that as long as the electrodes can achieve the above-mentioned functions and are electrically insulated from one another, the present invention doesn't limit the arrangement and the number of the electrodes. For example, the shape of the sensing electrode 123 can be arbitrary as long as it is capable of forming an electric loop with the measurement device, and additional electrodes can be added to accommodate various application needs.

The insulating layer 130 is disposed on the conductive layer 120, and includes an opening 135 to expose a part of the insulating substrate 110. It's sufficient for the opening 135 to expose part of the working electrode 121 and part of the reference electrode 122. The present invention is not limited to the shape of the opening 135. Besides, the insulating layer 130 also exposes another part (i.e. the connection region 128) of the conductive layer 120 so that the conductive layer 120 can electrically connect to a measurement device. The material of the insulating layer 130 can include but is not limited to: PVC insulating tape, PET insulating tape, thermal drying insulating paint or ultraviolet drying insulating paint.

The cover 150 is disposed on the insulating layer 130 and covers the opening 135. A sampling space (i.e. reaction area) with capillary attraction is formed between the insulating substrate 110 and the cover 150, which allows a sample to enter into the reaction area in the direction indicated by the arrow shown in FIG. 1. When the area of the sampling space is fixed, its volume depends on the thickness of the insulating layer 130. Generally, the thickness of the insulating layer 130 is between 0.005 and 0.3 millimeter, but not limited thereto. Regarding to manufacturing process, an insulating layer 130 with a precut opening 135 can be disposed above the insulating substrate 110 and the conductive layer 120. Alternatively, the insulating layer 130 can be directly formed above part of the insulating substrate 110 and the conductive layer 120 by a printing method, skipping areas of the opening 135 and the connection exposed region 128.

The biochemical test strip 100 of the present invention further includes a reaction layer 140 disposed within the opening 135, which has the ability to identify a specified organism material or signal. The material of the reaction layer 140 can be varied with types of the sample, such as an oxidoreductase or an electronic mediator, for reacting with the sample. Generally, the reaction layer 140 should at least cover part of the working electrode 121.

The cover 150 of the present invention can be transparent or translucent material, so that the users can check whether the sample has been disposed on the reaction area in order to avoid a false result. The lower surface of the cover 150 close to the reaction area can be coated with a hydrophile material to enhance the capillary action on the inner surface of the reaction area, whereby the sample can be conducted to the reaction area more quickly and efficiently. In another embodiment, instead of being coated on the lower surface of the cover 150, the hydrophile material, such as cellulose, carboxymethyl cellulose, methylcellulose, or other similar material, is added into the reaction layer to facilitate the capillary action. The cover 150 further includes a vent 155 corresponding to the opening 135 for expelling the air inside the reaction area to enhance the capillary action. Generally, the vent 155 is near the end side of the reaction area. The present invention is not limited to the shape of the vent 155. For example, the shape of the vent 155 can be circle, ellipse, rectangle, a rhombus, etc.

FIGS. 2-4 illustrate the biochemical test strips 200, 300, and 400 respectively according to different embodiments of the present invention, and it should be noted that the insulating layer and the cover for each biochemical test strip are not shown for purposes of clarity. Referring to FIG. 2, the biochemical test strip 200 includes an insulating substrate 210, a working electrode 221, a reference electrode 222, a sensing electrode 223, and an open-circuit part 226 located in the connecting region 228, wherein the working electrode 221, the reference electrode 222, and the sensing electrode 223 are insulated from one another. In this embodiment, in response to the location of the open-circuit part 226, the locations where the sensing electrode 223 is supposed to electrically connect with the measurement device are 229a and 229b. The configuration of the biochemical test strip 200 in FIG. 2 is similar to the biochemical test strip 100 in FIG. 1, except that the location of the open-circuit part 226 on the sensing electrode 223 is different from the location of the open-circuit part 126 on the sensing electrode 123. Because of the different locations of the open-circuit parts 126 and 226, the biochemical test strips 100 and 200 have corresponding specific measurement devices. For example, when the biochemical test strip 200 is inserted into a measurement device corresponding to the biochemical test strip 100, the sensing electrode 223 may be unable to form an electric loop with the measurement device due to the open-circuit part 226 of the biochemical test strip 200 so that the measurement device will not be initiated.

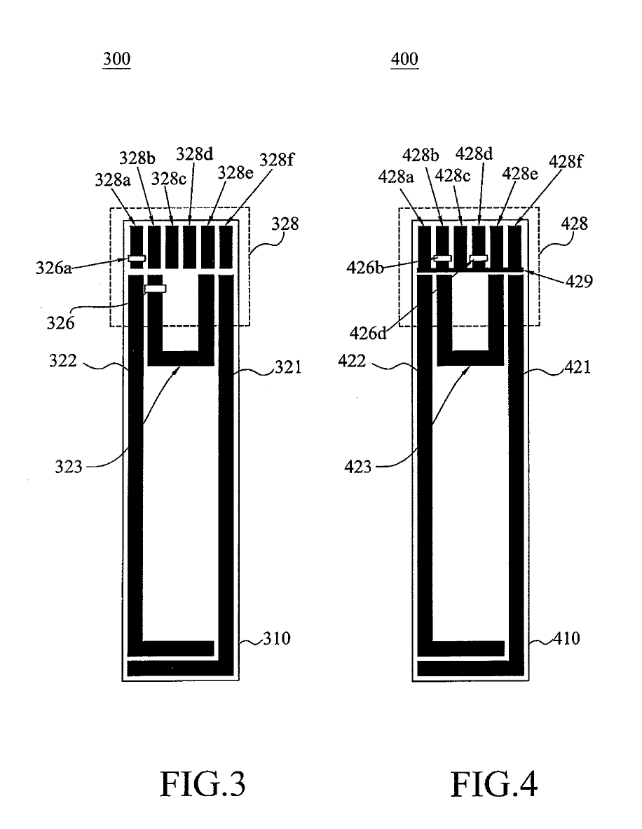

Referring to FIG. 3, the biochemical test strip 300 includes an insulating substrate 310, a working electrode 321, a reference electrode 322, a sensing electrode 323, and six identifying elements 328a, 328b, 328c, 328d, 328e, and 328f, wherein the working electrode 321, the reference electrode 322, and the sensing electrode 323 are insulated from one another. Within the connecting region 328, there are two open-circuit parts 326 and 326a located on the sensing electrode 323 and the identifying element 328a respectively. It should be noted that each of the identifying elements 328a-f can be selectively destructed to generate different identification codes, which enable a measurement device to recognize the type of the biochemical test strip. In this embodiment, the electrical properties of the sensing electrode 323 and the identifying element 328a are damaged by forming the open-circuit parts 326 and 326a thereon by, such as, a laser etching process, as shown in FIG. 3.

Referring to FIG. 4, the biochemical test strip 400 includes an insulating substrate 410, six identifying elements 428a, 428b, 428c, 428d, 428e, and 428f, a linking unit 429, a working electrode 421, a reference electrode 422, a sensing electrode 423, wherein the working electrode 421, the reference electrode 422, and the sensing electrode 423 are insulated from one another. Within the connecting region 428, there are two open-circuit parts 426b and 426d located on the identifying element 428b and 428d respectively. One side of the linking unit 429 connects to one end of each of the six identifying elements 428a, 428b, 428c, 428d, 428e, and 428f, which forms a parallel structure, for providing a common ground for these six components. In this embodiment, some electrical properties of the identifying elements 428b and 428d are damaged by forming the open-circuit parts 426b and 426d thereon by, such as, a laser etching process, as shown in FIG. 4.

When being inserted into a measurement device, because the damaged identifying elements (such as the identifying element 328a in FIG. 3 and the identifying elements 428b and 428d in FIG. 4) can not be electrically connected with the measurement device, the biochemical test strip 300 or 400 can be identified and measured with corresponding correction parameters or modes by the measurement device. Although each of the biochemical test strips shown in FIGS. 3 and 4 has six identifying elements, the present invention is not limited to the number of the identifying elements. It should be understood that the number and the location of the identifying elements and the open-circuit parts thereon can be altered by the designer according to practical applications to compose various identification codes. For example, 2^{N}-1 identification codes are available for a biochemical test strip with N identifying elements. In other words, each of identifying elements has two possible statuses: with and without an open-circuit part, so that multiple identification codes can be composed according to the statuses and the locations of the identifying elements. Accordingly, the biochemical test strips from different batches or with different functions can be characterized by the identifying elements.

FIGS. 5A-5D are illustrative diagrams showing the connectors 512, 522, 532, and 542 of the biochemical test strips 100, 200, 300, and 400 in FIGS. 1-4 respectively. Typically, the biochemical test strip can be electrically connected to a measurement device (such as a measurement device 630 shown in FIG. 6) through a connector (such as a connector 640 shown in FIG. 6) for running various tests. In one embodiment, the connector of the present invention includes at least a plurality of connecting terminals respectively corresponding to the electronic elements on the biochemical test strip, and by virtue of electrical coupling between the connecting terminals and the electronic elements on the biochemical test strip, the measurement device can receive corresponding signals from the biochemical test strip.

Referring to FIG. 5A, the connector 512 includes a measurement terminal 515w, a reference terminal 515z, and two sensing terminals 515x and 515y, which correspond to the working electrode 121, the reference electrode 122, and two ends of the [symbol] -shaped sensing electrode 123 of the biochemical test strip 100 respectively. Because there is an open-circuit part 126 formed in the connection region 128, the sensing terminal 515x is repositioned, for example, upwardly to the position shown in FIG. 5A, such that an electric loop used to initiate the measurement device can be formed among the sensing electrode 123 and the sensing terminals 515x and 515y. Referring to FIG. 5B, the connector 522 includes a measurement terminal 525w, a reference terminal 525z, and two sensing terminals 525x and 525y, which correspond to the working electrode 221, the reference electrode 222, and two ends of the [symbol] -shaped sensing electrode 223 of the biochemical test strip 200 respectively. Similarly, in response to the presence of the open-circuit part 226 in the connection region 228, the location of the sensing terminal 525y is adjusted upwardly to correspond to a location above the open-circuit part 226, as shown in FIG. 5B, such that an electric loop can be formed among the sensing electrode 223 and the sensing terminals 525x and 525y. Comparing FIG. 5A and FIG. 5B, when the biochemical test strip 100 is inserted into a measurement device having the connector 522 in FIG. 5B, the sensing terminal 525x will contact with the open-circuit part 126 of the biochemical test strip 100, such that the measurement device can't be initiated because no loop is formed among the sensing electrode 123 and the sensing terminals 525x and 525y.

Referring to FIG. 5C, the connector 532 includes a measurement terminal 535w, a reference terminal 535z, and two sensing terminals 535x and 535y, which correspond to the working electrode 321, the reference electrode 322, and two ends of the [symbol] -shaped sensing electrode 323 of the biochemical test strip 300 respectively. The connector 532 further includes six identifying terminals 535a, 535b, 535c, 535d, 535e, and 535f, which correspond to the six identifying elements 328a, 328b, 328c, 328d, 328e, and 328f respectively. In response to the presence of the open-circuit part 326 in the connection region 328, the location of the sensing terminal 535y is adjusted upwardly, such that a loop can be formed among the sensing electrode 323 and the sensing terminals 535x and 535y. On the other hand, there is no electrical connection between the identifying terminal 535a and the identifying element 328a due to the open-circuit part 326a, whereby the measurement device can recognize the type of the biochemical test strip 300.

Referring to FIG. 5D, the connector 542 includes a measurement terminal 545w, a reference terminal 545z, and two sensing terminals 545x and 545y, which correspond to the working electrode 421, the reference electrode 422, and two ends of the [symbol] -shaped sensing electrode 423 of the biochemical test strip 400 respectively. The connector 542 further includes six identifying terminals 545a, 545b, 545c, 545d, 545e, and 545f, and a ground terminal 545g, which correspond to the six identifying elements 428a, 428b, 428c, 428d, 428e, 428f, and the linking unit 429 respectively. In this embodiment, due to the open-circuit parts 426b and 426d, there is no electrical connection either between the identifying terminal 545b and identifying elements 428b or between the identifying terminal 545d and the and 428d, whereby the measurement device can recognize the type of the biochemical test strip 400.

FIG. 6 is a block diagram of a biochemical test system 600 according to an embodiment of the present invention, including a biochemical test strip 610 and a measurement device 630. The biochemical test strip 610 includes a working electrode 621, a reference electrode 622, a sensing electrode 623, six identifying elements 628a, 628b, 628c, 628d, 628e, 628f and a linking unit 629. One side of the linking unit 629 connects with one end of each of identifying elements 628a-f, and the other side of the linking unit 629 connects with the sensing electrode 623. The linking unit 629 functions as a common ground. In this embodiment, the identifying element 628c and one end of the sensing electrode 623 are electrically damaged by, such as, a laser etching process, i.e. there are two open-circuit parts 626 and 626c formed on the sensing electrode 626 and the identifying elements 628c within the connection region 628 of the biochemical test strip 610. In another embodiment, the linking unit 629 can further connect with the reference electrode 622 (not shown) for providing the common ground for the reference electrode 622.

The measurement device 630 includes a connector 640 and a microprocessor 650 coupled to the connector 640. The digital data 655, for example, testing parameters, detection modes or other information, are stored in the microprocessor 650. The working electrode 621, the reference electrode 622, the sensing electrode 623, the linking unit 629, and the identifying elements 628a-628f are connected to the measurement 630 through the connector 640 respectively. In this embodiment, the connector 640 includes a measurement terminal 645w, a reference terminal 645z, a sensing terminal 645y, and a ground terminal 645g, which correspond to the working electrode 621, the reference electrode 622, the sensing electrode 623, and the linking unit 629 of the biochemical test strip 610 respectively. Because one end of the sensing electrode 623 is connected to the linking unit 629 for common ground and the ground terminal 645g is also connected to the linking unit 629, the ground terminal 645g and the sensing electrode 623 are electrically connected with each other. In response to the location of the open-circuit part 626, the location of the sensing terminal 645y is adjusted upwardly, such that a loop can be formed among the sensing electrode 623 and the terminals 645y and 645g. The connector 640 further includes six identifying terminals 645a, 645b, 645c, 645d, 645e, and 645f, which correspond to the six identifying elements 628a-f respectively.

When the biochemical test strip 610 is inserted into the measurement device 630, due to the presence of the open-circuit part 626, the measurement device 630 can be initiated only on the condition of having a connector with terminals arranged like the terminals of the connector 640 shown in FIG. 6. For example, if the sensing electrode 623 is not damaged (i.e. absence of the open-circuit part 626), the biochemical test strip 610 can be used with the connector 542 shown in FIG. 5D, which can initiate the measurement device by the electric loop formed among the sensing electrode 623 and the sensing terminals 545x and 545y. However, if the biochemical test strip 610 is inserted into the connector 542 in FIG. 5D while the open-circuit part 626 is present, no electric loop can be formed among the sensing electrode 623 and the sensing terminals 545x and 545y and therefore the measurement device can not be initiated. In other words, the biochemical test strips having different number and location of the one open-circuit parts are corresponding to different measurement devices with different structures of the connectors.

Furthermore, since the identifying elements 628a-628f have different statuses respectively (i.e. be damaged or not), the electrical connection between the connector 640 and the identifying elements 628a-628f has different possible configurations, and signals corresponding to the electrical connection can be generated and transmitted to the microprocessor 650. For example, since the damaged identifying element 628c is unable to electrically connect with the connector 640, an open-circuit signal corresponding to the open-circuit part 626c will be generated and transmitted to the microprocessor 650. After receiving the signal, the microprocessor 650 can select testing parameters or a test mode from the digital data 655 corresponding to the signal for executing the test procedure. The measurement device 630 can further include a monitor 670 for displaying each measurement result and a power source 660 for supplying power to the system. In another embodiment, the monitor 670 and the power source 660 can be external devices, not included within the measurement device 630.

The present invention can control whether a measurement device can be initiated by a particular test strip by selectively damaging a sensing electrode formed on this particular test strip. The identifying elements of the present invention are provided for identification and to designate the data stored in the measurement device. That is, one of the plurality of testing parameters, detection modes, or other information corresponding to the configuration of the identifying elements can be selected by the measurement device to perform the test procedure. To sum up, the biochemical test strip and system disclosed in the present invention not only achieve the goal to avoid the use of code card, but also reduce the production cost.

FIG. 7 is a flow chart for manufacturing a biochemical test strip according to an embodiment of the present invention. First, in step S700, an insulating substrate is provided. Then, in step S710, a conductive layer is formed on the insulating substrate by coating a conductive material. The conductive layer includes a plurality of electronic elements insulated from each other, and one end of the conductive layer is formed as a connection region. In one embodiment, the plurality of electronic elements may include a working electrode, a reference electrode, and a sensing electrode. In another embodiment, the plurality of electronic elements may include a working electrode, a reference electrode, and a plurality of identifying elements which is configured to enable a measurement device to recognize the type of the biochemical test strip. In still another embodiment, the plurality of electronic elements may include a working electrode, a reference electrode, a sensing electrode, and a plurality of identifying elements. Then, in step S720, at least one open-circuit part is formed on at least one of the plurality of the electronic elements within the connection region by applying an electrical damage on a small part of one or more electronic elements within the connection region. Typically, the method of forming the open-circuit part can be a mechanical abrasion method or a chemical etching method, and preferably, be a laser etching method which is capable of precisely and finely making an electrical damage on the electronic element. For example, the open-circuit part can be formed by electrolysis, exposure and development, planer process, drilling process, or other similar method. In one embodiment, the open-circuit part can be formed on the sensing electrode only. In another embodiment, the open-circuit part(s) can be formed on one or more of the identifying elements. In still another embodiment, the open-circuit parts can be formed on both of the sensing electrode and at least one of the identifying elements. Next, in step S730, an insulating layer having an opening is formed on the conductive layer to partially cover the insulating substrate. A part of the conductive layer exposed by the opening of the insulating layer is defined as a reaction area. In addition, the insulating layer also exposes the connection region of the conductive layer. Then, in step S740, a reaction layer with the ability to identify specified organism material or signal is disposed on the reaction area. Next, in step S750, a cover is disposed on the insulating layer to cover the opening, whereby a sampling space with capillary attraction is formed between the insulating substrate and the cover.

The above illustration is for preferred embodiments of the present invention, and is not limited to the claims of the present invention. Equivalent amendments and modifications without departing from the spirit of the invention should be included in the scope of the following claims.

1. A biochemical test strip, comprising:
an insulating substrate;
a conductive layer disposed on the insulating substrate and having a plurality of electronic elements, wherein one end of the conductive layer is formed as a connection region; and
at least one open-circuit part disposed on at least one of the plurality of the electronic elements within the connection region;
wherein a type of the biochemical test strip is determined by number and location of the at least one open-circuit part.

2. The biochemical test strip according to claim 1, wherein the plurality of the electronic elements comprise a working electrode, a reference electrode, and a sensing electrode insulated from one another, and the at least one open-circuit part is located on the sensing electrode.

3. The biochemical test strip according to claim 1, wherein the plurality of the electronic elements comprise a working electrode, a reference electrode, and a plurality of identifying elements insulated from one another, and the at least one open-circuit part is located on at least one of the plurality of identifying elements, and wherein an identification code of the biochemical test strip is determined by number and location of the at least one open-circuit part on the plurality of identifying elements.

4. The biochemical test strip according to claim 1, wherein the plurality of the electronic elements comprise a plurality of identifying elements and further comprise a working electrode, a reference electrode, and a sensing electrode insulated from one another, and the at least one open-circuit part is located on at least one of the plurality of identifying elements and the sensing electrode, and wherein an identification code of the biochemical test strip is determined by number and location of the at least one open-circuit part on the plurality of identifying elements.

5. The biochemical test strip according to claim 1, wherein the plurality of the electronic elements comprise a working electrode, a reference electrode, a plurality of identifying elements and a linking unit, and the at least one open-circuit part is located on at least one of the plurality of identifying elements;
an identification code of the biochemical test strip is determined by number and location of the at least one open-circuit part on the plurality of identifying elements; and
wherein a first side of the linking unit is connected to one terminal of each of the plurality of identifying elements to provide a common ground.

6. The biochemical test strip according to claim 5, wherein the plurality of the electronic elements further comprise a sensing electrode, and the at least one open-circuit part is located on at least one of the plurality of identifying elements and the sensing electrode.

7. The biochemical test strip according to claim 6, wherein a second side of the linking unit is connected to one terminal of the sensing electrode.

8. The biochemical test strip according to claim 7, wherein the second side of the linking unit is further connected to one terminal of the reference electrode.

9. The biochemical test strip according to claim 3, wherein the plurality of identifying elements are N identifying elements, and the N identifying elements along with the number and the location of the at least one open-circuit part generate 2^{N}-1 identification codes.

10. The biochemical test strip according to claim 1, wherein the at least one open-circuit part is formed by damaging a part of at least one of the plurality of the electronic elements by a laser etching process.

11. The biochemical test strip according to claim 10, wherein the at least one open-circuit part either penetrates or does not penetrate the insulating substrate.

12. A biochemical test system, comprising:
a biochemical test strip, comprising an insulating substrate, a conductive layer disposed on the insulating substrate, and at least one open-circuit part, wherein the conductive layer has a plurality of electronic elements, one end of the conductive layer is formed as a connection region, and the at least one open-circuit part is disposed on at least one of the plurality of the electronic elements within the connection region, and wherein a type of the biochemical test strip is determined by number and location of the at least one open-circuit part; and
a measurement device, comprising a microprocessor and a connector, wherein the connector comprises a plurality of connecting terminals respectively corresponding to the plurality of electronic elements and the at least one open-circuit part, the plurality of connecting terminals are configured to be coupled to the connection region for receiving a signal corresponding to the type of the biochemical test strip, and the microprocessor is coupled to the connector for receiving the signal from the connector.

13. The biochemical test system according to claim 12, wherein the plurality of the electronic elements comprise a working electrode, a reference electrode, and a sensing electrode insulated from one another, the at least one open-circuit part is located on the sensing electrode, and an electric loop is formed among two of the plurality of the connecting terminals of the connector and the sensing electrode.

14. The biochemical test system according to claim 12, wherein the plurality of the electronic elements comprise a working electrode, a reference electrode, and a plurality of identifying elements insulated from one another, and the at least one open-circuit part is located on at least one of the plurality of identifying elements;
wherein an identification code of the biochemical test strip is determined by number and location of the at least one open-circuit part on the plurality of identifying elements; and
wherein the plurality of identifying elements respectively correspond to the plurality of connecting terminals, and the at least one open-circuit part is configured to break an electrical connection between at least one of the plurality of the identifying elements and the corresponding connecting terminal thereof.

15. The biochemical test system according to claim 12, wherein the plurality of the electronic elements comprise a plurality of identifying elements and further comprise a working electrode, a reference electrode, and a sensing electrode insulated from one another, and the at least one open-circuit part is located on at least one of the plurality of identifying elements and the sensing electrode;
wherein an electric loop is formed among two of the plurality of the connecting terminal and the sensing electrode;
wherein an identification code of the biochemical test strip is determined by number and location of the at least one open-circuit part on the plurality of identifying elements; and
wherein the plurality of identifying elements respectively correspond to the plurality of connecting terminals, and the at least one open-circuit part is configured to break an electrical connection between at least one of the plurality of the identifying elements and the corresponding connecting terminal thereof.

16. The biochemical test system according to claim 12, wherein the plurality of the electronic elements comprise a working electrode, a reference electrode, a plurality of identifying elements and a linking unit, and the at least one open-circuit part is located on at least one of the plurality of identifying elements;
an identification code of the biochemical test strip is determined by number and location of the at least one open-circuit part on the plurality of identifying elements;
wherein the plurality of identifying elements respectively correspond to the plurality of connecting terminals, and the at least one open-circuit part is configured to break an electrical connection between at least one of the plurality of the identifying elements and the corresponding connecting terminal thereof; and
wherein one side of the linking unit is connected to one terminal of each of the plurality of identifying elements to provide a common ground.

17. The biochemical test system according to claim 13, wherein the connector transmits a control signal corresponding to the electric loop to the microprocessor to enable the microprocessor to initiate the measurement device.

18. The biochemical test system according to claim 12, wherein the at least one open-circuit part is formed by a laser etching process.

19. The biochemical test system according to claim 12, wherein a plurality sets of correction parameters are stored in the microprocessor, and the microprocessor selects one set of the correction parameters to calibrate the biochemical test system according to the received signal.

20. The biochemical test strip according to claim 12, wherein a plurality of modes are stored in the microprocessor, and the microprocessor selects one mode for execution according to the received signal.

21. A measurement device for use with a biochemical test strip, wherein the biochemical test strip comprises an insulating substrate, a conductive layer disposed on the insulating substrate, and at least one open-circuit part, wherein the conductive layer has a plurality of electronic elements, one end of the conductive layer is formed as a connection region, and the at least one open-circuit part is disposed on at least one of the plurality of the electronic elements within the connection region, and wherein a type of the biochemical test strip is determined by number and location of the at least one open-circuit part, the measurement device comprises:
a connector comprising a plurality of connecting terminals corresponding to the plurality of electronic elements and the at least one open-circuit part respectively, wherein the plurality of connecting terminals are configured to be coupled to the connection region for receiving a signal corresponding to the type of the biochemical test strip; and
a microprocessor coupled to the connector for receiving the signal from the connector.

22. The measurement device according to claim 21, wherein the plurality of the electronic elements comprise a working electrode, a reference electrode, a sensing electrode, and a plurality of identifying elements insulated from one another, and the at least one open-circuit part is located on at least one of the plurality of identifying elements and the sensing electrode;
wherein an electric loop is formed among two of the plurality of the connecting terminals and the sensing electrode;
wherein an identification code of the biochemical test strip is determined by number and location of the at least one open-circuit part on the plurality of identifying elements; and
wherein the plurality of identifying elements respectively correspond to the plurality of connecting terminals, and the at least one open-circuit part is configured to break an electrical connection between at least one of the plurality of the identifying elements and the corresponding connecting terminal thereof.

23. The measurement device according to claim 21, wherein the at least one open-circuit part is formed by a laser etching process.
